SEQUENCE LISTING (4 sequences)

SEQ ID NO: 1 - DNA, 25 bp, synthetic construct
  source 1..25: mol_type = other DNA
gtttttgagctcgaagttcagctgc

SEQ ID NO: 2 - DNA, 33 bp, synthetic construct
  source 1..33: mol_type = other DNA
gtttttactagttttcagttccagtttagtacc

SEQ ID NO: 3 - DNA, 720 bp, synthetic construct
  source 1..720: mol_type = other DNA
gaagttcagctgctgcaatctggtccagaactggagaaaccgggtgcgtctgttatgatctcttgtaaagcatctggctccagcttcaccggttacaacatgaactgggttcgtcagaacatcggcaaaagcctggaatggatcggcgcgatcgacccatactatggcggcacctcttacaaccagaaattcaaaggtcgcgctacgctgactgtcgataaatcttcttccaccgcatatatgcacctgaaaagcctgacttctgaagactctgccgtatactactgcgttagcggtatggaatactggggtcagggtacttccgttaccgtttctggtggtggcggttctggtggtggtggttctggtggtggcggtagcgatgttgtgatgacccagacccctctgagcctgccggtatccctgggtgaccaggcttctatctcttgccgttcctctcagtctctggttcatcgcaatggtaacacctatctgcactggtatctgcagaagccgggtcagtccccaaaactgctgattcataaagtttctaaccgtttctctggtgttccggaccgtttctccggttctggcagcggtactgatttcaccctgaaaattagccgcgtcgaagctgaagacctgggcgtgtacttttgttctcagtccacccacgtgccgccactgacctttggtgctggtaccaaactggaactgaaa

SEQ ID NO: 4 - protein, 240 aa, synthetic construct
EVQLLQSGPELEKPGASVMISCKASGSSFTGYNMNWVRQNIGKSLEWIGAIDPYYGGTSYNQKFKGRATLTVDKSSSTAYMHLKSLTSEDSAVYYCVSGMEYWGQGTSVTVSGGGGSGGGGSGGGGSDVVMTQTPLSLPVSLGDQASISCRSSQSLVHRNGNTYLHWYLQKPGQSPKLLIHKVSNRFSGVPDRFSGSGSGTDFTLKISRVEAEDLGVYFCSQSTHVPPLTFGAGTKLELK

(19) United States
(12) Patent Application Publication
(10) Pub. No.: US 2026/0337582 A1
(43) Pub. Date: Oct. 29, 2026

(54) BACTERIOPHAGES AND USES THEREOF FOR TARGETING GD2-EXPRESSING TUMOR CELLS

(71) Applicant: ALMA MATER STUDIORUM - UNIVERSITA' DI BOLOGNA, Bologna (IT)

(72) Inventors: Alberto DANIELLI, Bologna (IT); Giovanni PERINI, Bologna (IT); Matteo CALVARESI, Bologna (IT); Piergiuseppe DE ROSA, Bologna (IT); Matteo DI GIOSIA, Bologna (IT); Roberto SAPORETTI, Bologna (IT); Luca ULFO, Bologna (IT); Paolo Emidio COSTANTINI, Bologna (IT); Suleman Khan ZADRAN, Bologna (IT)

(21) Appl. No.: 19/483,681

(22) PCT Filed: May 16, 2024
(86) PCT No.: PCT/EP2024/063519

§ 371 Date: Nov. 12, 2025

(30) Foreign Application Priority Data
May 16, 2023 (IT) 102023000009903

(51) Int. Cl. C12N 7/00; A61K 35/76; A61K 41/00; A61K 45/06; A61P 35/00; C07K 16/30

(52) U.S. Cl. CPC C12N 7/00; A61K 35/76; A61K 41/0057; A61K 45/06; A61P 35/00; C07K 16/3084; C07K 2317/54; C07K 2317/55; C07K 2317/622; C12N 2795/14121; C12N 2795/14132

(57) ABSTRACT

The present invention relates to a bacteriophage, or a composition that comprises it, comprising and/or expressing at least one ligand targeting disialoganglioside (GD2) and to its medical or diagnostic uses.

### FIELD OF THE INVENTION

The present invention relates to a bacteriophage, or a composition that comprises it, comprising and/or expressing at least one ligand targeting disialoganglioside (GD2), and its medical uses.

### PRIOR ART

Neuroblastoma (NB) is one of the most common pediatric neuroendocrine tumors originating from the neural crest-derived sympathoadrenal precursor cells and accounts 15% of total pediatric cancer mortalities (Coughlan et al., 2017). The patients of these neuroendocrine tumors bear distinguished characteristics including early age of onset, heterogenous clinical behavior, high metastatic tendency and genetic alterations (Matthay et al., 2016; Ciaccio et al., 2021). These features make it an aggressive malignancy leading to poor prognosis (Louis & Shohet, 2015). Recent treatments for High-Risk Neuroblastoma rely on conventional chemotherapy, radiation, autologous stem-cell transplant, surgical resections, and immunotherapy. Despite the rigorous treatment strategies, the overall 5 years survival rate is about 50% and these implemented medications have substantial late adverse effects including cognitive impairments, hearing loss, endocrinopathies and failure of ovaries (Friedman & Henderson, 2018; Portwine et al., 2016).

During oncological treatments, various therapeutics modalities are often combined to be more effective compared to single treatments (Meng et al., 2020).

Gangliosides are modified sphingolipids that are abundantly expressed in many cancer cells and relatively very low expressed in healthy tissues. Disialoganglioside (GD2) is a tumor-associated antigen expressed on the surface of neuroblastoma (NB) cells (5-10 million molecules/cell) and in tissue of the patients regardless of the stage of disease (Nazha et al., 2020; Zhang et al., 2019). Poor prognosis of NB patients and rapid progression is associated with circulating tumors (Berois & Osinaga, 2014). The specific role of the GD2 is still elusive, nevertheless it is thought to enhance tumor cell proliferation, mediate cell adhesion, motility, invasion and migration (Paret et al., 2022). Of interest, based on the level of expression, therapeutic effect, immunogenicity and level of antigen specificity, the U.S national institute ranked GD2 twelfth among 75 potential therapeutic targets (Park & Cheung, 2020). Notably, GD2 expressing tumors have been targeted with multi-modal therapies by using anti-GD2 monoclonal antibodies (Voeller & Sondel, 2019) and recently, pre-clinical studies on CAR-T cells (Prapa et al., 2021) have been published. Nevertheless, these therapies have shown insufficient outcomes with high costs, poor specificity and inadequate penetration of the antibody (Bonaventura et al., 2019; Melero et al., 2007).

Bacteriophages are viruses that infect and replicate only in bacterial cells and are unable to reproduce or infect eukaryotic cells. They are relatively homogeneous in size and morphology and exhibit excellent biocompatibility along with a good resistance to high temperatures, pH, proteolytic and nucleolytic enzymes (Principi et al., 2019). M13 phage has emerged as a captivating nanobioscaffold because of its genetically customizable capsid proteins and the ability to self-assemble into hierarchical structures (Chang et al., 2023). It allows researcher to use it in widespread biomedical applications which provides quick, effective and low cost production for exploring receptor binding sites, protein-protein interaction, epitopes, mimotopes and functional regions of antigens (Yeoh et al., 2022; Alfaleh et al., 2022). M13 phages can also be engineered into delivery vectors, as they can sustain versatile genetic display and chemical conjugation. The phage capsid is formed by 5 proteins, where pVIII is the major protein (2700 copies) and 4 minor proteins with 5 copies of each pIII, pVI, pVII and pIX. M13 has recently been applied as vector for photodynamic therapy (PDT) (Ulfo et al., 2022; Bortot et al., 2022). PDT has been investigated extensively for the treatment of various types of cancer and comprises three main components i.e. photosensitizer (PS), light and oxygen (Allison, 2014; Moore et al., 2009). In general, the PS can be excited with light at a given wavelength, generating Reactive oxygen species ROS (free radical, Singlet oxygen, Triplet oxygen) which eventually lead to the killing of cancer cells directly or to vascular destruction, or immune system activation against the tumors (Casas et al., 2011; Dolmans et al., 2003). PDT photosensitized oxidation of biomolecules involves two mechanisms. I) In direct-contact reaction the light energy elapses from excited PS to biomolecules initiating radical chain reaction and culminates in specific destruction of the biomolecules. II) the excited energy passed to molecular oxygen (³O₂) produces singlet oxygen (102), which is remarkably hydrophilic leading to attack membrane, protein, and DNA (Agostinis et al., 2011). Moreover, the autofluorescence of the PS enables to stain the early malignancies, pre-cancer lesion, tumour margin and residual dysplastic tissues after surgical resection of tumours. The therapeutic and diagnostic features of PS defines them as theragnostic agents (Josefsen & Boyle, 2012). However, accumulation of PS outside the tumor lesion can induce organ toxicity and there is a strong need to deliver and preferentially accumulate PS in the tumor (Xu et al., 2020). In this context, nanotechnology may aid the engineering of novel platforms to deliver drugs and therapeutic agents against NB cells (Cossu et al., 2015; Joseph et al., 2023).

In particular, there is an unmet need to provide a therapy for GD2 expressing tumors.

### SUMMARY OF THE INVENTION

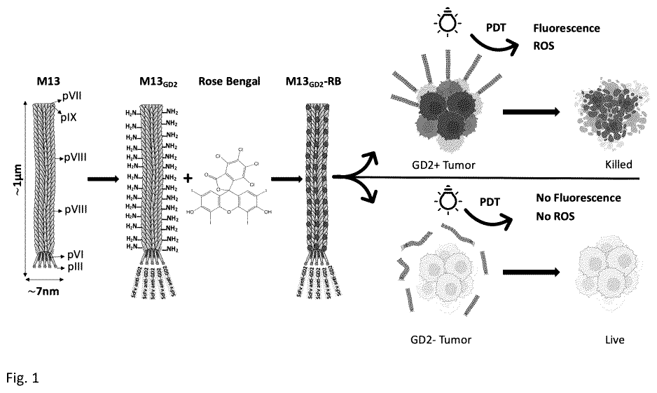

Gangliosides GD2 are extensively expressed on the neuroblastoma cell surface. They represent an attractive targeted tumour-associated antigen. Here inventors used an orthogonal approach (genetic/chemical) to target GD2 with an engineered M13 filamentous phage vector, demonstrating efficient targeting of the vector and lethal photosensitization of neuroblastoma cell lines upon light irradiation when the M13 vector was rigged with photosensitisers. In particular, the M13 phage was refactored (M13_{GD2}) to display a 14G2a-based Single chain fragment Variable ScFv (Lv-Hv) against GD2 in pentavalency. The M13_{GD2} major capsid protein was then conjugated with hundreds of Rose Bengal photosensitizers without impairing the specific targeting of the vector. The M13_{GD2} vector was also able to target tumor spheroids of GD2-positive cells and surprisingly to penetrate deep into the spheroids. Interestingly, upon irradiation with low power white light, M13_{GD2}RB triggered lethal photosensitization of GD2-positive cells at picomolar concentrations, whereas the GD2-negative cell were unaffected. Moreover, M13_{GD2}RB displayed no dark toxicity in GD2 positive and GD2 negative NB cells (FIG. 1). The maintenance of high selectivity, despite the high number of RB molecules conjugated to the phage indicates that surprisingly the targeting molecules were not affected by decoration with RB. This represents an advantage over known technologies of the art involving direct conjugation of photosensitizers to the targeting molecules (e.g. antibodies, scFv, etc), since it is known to the person skilled in the art that these lose affinity for their targets when conjugated with high numbers of effector (sensitizer) molecules (Pelegrin et al., 1991; van Dongen et al., 2004).

Therefore an object of the invention is a bacteriophage, or a composition that comprises it, comprising and/or expressing at least one ligand targeting disialoganglioside (GD2), wherein the bacteriophage is conjugated with at least one molecule selected from: a photosensitiser, a chemotherapeutic drug, a lipid, a fluorophore, a reporter, a contrast agent, a radionuclide, a boronated molecule, a catalyst, a polymer, a polysaccharide, a nucleic acid, a surfactant, a positively charged molecule (e.g., methyl ammonium containing molecules), an amphiphilic molecule, an anti-microbial glycan, or Toll-Like Receptor agonists.

Preferably, the bacteriophage is selected from the M13, fd phage, Ff phage, T4, Qβ and MS2 phage. The bacteriophage is preferably the M13 phage, more preferably M13K07.

Preferably, the ligand is selected from: an antibody or an antigen binding fragment thereof, a receptor, a nanobody, a protein, a peptide and combinations thereof.

Preferably, the ligand is encoded by a nucleic acid fused to a nucleotide sequence encoding a coat protein of the bacteriophage, preferably the coat protein is the protein pIII.

Preferably, the antigen-binding fragment is a single chain fragment variable (scFv), Fab, Fab′, or F(ab′)2, preferably it is an anti-GD2 ScFv, preferably said ScFv comprises the heavy chain variable region (VH) and light chain variable region (VL) of the 14.G2a antibody (or 14G2a antibody).

Preferably the VH is located at N-terminal to the VL and linked to the VL via a linker peptide.

Preferably, the bacteriophage is genetically modified by fusing at least one ligand to the pIII protein of the bacteriophage.

Preferably, at least one ligand is fused to the C-terminal domain of the pIII protein of the bacteriophage.

Preferably, the ligand is a scFv.

Preferably the ligand is an anti-GD2 ScFv, preferably said ScFv comprises the heavy chain variable region (VH) and/or light chain variable region (VL) of the 14.G2a antibody.

Preferably, the single-chain fragment variable (ScFv) or the single-chain fragment variable (ScFv) sequence comprises a sequence having at least 80% of identity to SEQ ID NO:4 or is encoded by a sequence having at least 80% of identity to SEQ ID NO:3 or by a sequence encoding a sequence having at least 80% of identity to SEQ ID NO:4.

Preferably, the bacteriophage comprises 1, 2, 3, 4, or 5 copies of the ligand. In a preferred embodiment, the bacteriophage comprises 5 copies of the ligand, preferably the ligand being an anti-GD2 ScFv. In an even more preferred embodiment, the bacteriophage comprises 5 copies of an anti-GD2 ScFv fused to the minor coat pIII protein at the phage distal end.

Preferably the photosensitizer is selected from: Bacteriochlorin, Phthalocyanines, Pheophorbide A and B, Bacteriopheophorbide, Naphthalocyanine, BODIPY, Anthracycline, Tetracycline, Fluoroquinolones, Phenothiazine, Perylene, Perylenequinone, Curcumin, Quinones, Anthraquinones, Fullerenes, Endohedral Metallofullerenes, Acridines, Isoquinole alkaloids, Xanthenes, Porphyrins, Chlorins and Cyanine, more preferably the photosensitizer is selected from: xanthens, preferably rose bengal, eosin, erythrosine B, fluorescein and rhodamine, porphyirins, preferably protoporphyirin IX, hematoporphyirin, porfimer sodium and ATX-70; chlorins, preferably chlorin e6, temoporfin and verteporfin; cyanines, preferably cyanine IR-806, indocyanine green and cyanine IR-780, and combinations thereof.

Preferably the photosensitiser is selected from Porphyrins, phthalocyanines, chlorins, bacteriochlorins, phenothiaziniums, phenazines, acridines, texaphyrins, cyanines, anthracyclins, pheophorbides, sapphyrins, fullerene, halogenated xanthenes, perylenequinonoid pigments, gilvocarcins, terthiophenes, benzophenanthridines, psoralens and riboflavin.

Preferably the at least one molecule is covalently linked to the bacteriophage. Alternatively, it is not covalently bound to the bacteriophage.

A further object of the invention is a pharmaceutical composition comprising the bacteriophage or the composition as described above or herein and at least one excipient, vehicle or diluent.

Another object of the invention is the bacteriophage or the composition or the pharmaceutical composition as defined above or herein for use as a medicament, preferably for use in the prevention and/or treatment or follow-up of a disease or disorder mediated by GD2.

Preferably the disease or disorder mediated by GD2 is a GD2 positive tumor or a tumor comprising at least one cell expressing GD2, preferably selected from the group of brain tumor, neuroblastoma, glioblastoma, medulloblastoma, retinoblastoma, astrocytoma, melanoma, B-cell lymphoma, small cell lung cancer, renal carcinoma, desmoplastic small round cell fibroma, osteosarcoma, Ewing's sarcoma, breast cancer, rhabdomyosarcoma, leiomyosarcoma, liposarcoma, fibrosarcoma, or soft tissue sarcoma.

Preferably the disease or disorder mediated by GD2 is a tumor which expresses or comprises at least one cell which expresses GD2.

The bacteriophage or the composition or the pharmaceutical composition as defined above or herein is preferably for use in the treatment of tumors, preferably GD2 positive tumors or tumors comprising at least one cell expressing GD2.

Preferably the tumor is selected from the group of brain tumor, neuroblastoma, glioblastoma, medulloblastoma, retinoblastoma, astrocytoma, melanoma, B-cell lymphoma, small cell lung cancer, renal carcinoma, desmoplastic small round cell fibroma, osteosarcoma, Ewing's sarcoma, breast cancer, rhabdomyosarcoma, leiomyosarcoma, liposarcoma, fibrosarcoma, or soft tissue sarcoma.

Another object of the invention is the bacteriophage or the composition as defined above or the pharmaceutical composition as defined above or herein for use in detection and/or imaging of cells or tissue expressing GD2. In this case, the phage is preferably coupled or conjugated with a fluorophore (e.g. Cy3, Cy5, FITC) or another reporter molecule.

The bacteriophage or the composition or the pharmaceutical composition is preferably in association or in combination with at least one an antitumoral agent, preferably an antibody, a chemotherapeutic agent, a radionuclide, a hormone therapy agent or an immune checkpoint inhibitor, and/or with a surgical treatment, preferably a treatment of a malignant tumor, more preferably before or after an operation to remove the malignant tumor.

Another object of the invention is an in vitro method of diagnosis of a disease wherein disease cells express GD2 or of detection and/or imaging of cells or tissue expressing GD2, comprising determining whether a cell from a subject binds to the bacteriophage or the composition as defined above.

### DETAILED DESCRIPTION OF THE INVENTION

Here inventors employ an orthogonal approach to use M13 phage as a targeting vector for GD2-positive NB cells and the present system provides strong evidence of specificity and sensitivity for GD2-positive cells, having no binding on GD2-negative cells, even in a mixed population of cell lines. Moreover, after conjugation of the retargeted phage with sensitizers the phage vector can induce lethal photosensitization of NB cells upon low-intensity light irradiation.

In the context of the present invention, the terms “bacteriophage” and “phage” are interchangeable and mean a virus that only parasitises bacteria, but not eukaryotic cells.

In the context of the present invention, the term “photosensitiser” means a molecule capable of generating reactive oxygen species (ROS) following irradiation with light.

In the context of the present invention, the term “photodynamic therapy (PDT)” means a non-invasive therapy that envisages the combined use of light and photosensitisers, which can generate reactive oxygen species (ROS).

In the context of the present invention the term “ligand” means an element capable of recognising and/or targeting and/or binding, preferably in a specific and/or selective manner, to an element expressed by a cell or a virus, as GD2.

GD2 is disclosed in PubChem ID 6450346.

The bacteriophage of the invention is preferably a recombinant bacteriophage or an engineered bacteriophage which comprises a nucleic acid sequence encoding a ligand that is capable of being expressed in the capsid coat, and which is also specific for a protein expressed on a target cell/tissue, as GD2, so as to enable targeted delivery thereto.

In the context of the present invention the ligand is therefore a tumor-targeting ligand, in particular GD2-positive tumor.

The bacteriophage may also comprise a transgene, which exerts a therapeutic effect on a target cell.

The bacteriophage is preferably a filamentous bacteriophage, such as Ff, Fd or M13.

The bacteriophage of the invention may also be used in an imaging method. That means that the phage can be coupled with a fluorophore (e.g. Cy3, Cy5, FITC) and used to target and detect the GD2 antigen on cells in vitro and in vivo

In the present invention, the conjugation takes place with methods known to the person skilled in the art; it is typically mediated by a covalent bond, or by a non-covalent bond to the bacteriophage capsid.

The ligand is adapted to favour binding of the phage to the target cell. In fact, in the present invention the phage is preferably engineered to express, on its capsid, the ligand capable of directing the phage towards a GD2-positive cell.

The present invention also relates to a composition comprising the phage as described above and excipients and/or additional substances and/or pharmaceutically accepted carriers.

According to a preferred embodiment of the invention, the composition is formulated for parenteral administration. In particular, the composition is formulated in liquid form, preferably in the form of a solution, emulsion or sterile suspension.

In one embodiment, the composition is in lyophilised form, to be reconstituted so as to obtain a liquid formulation.

The phage of the present invention, thanks to the presence of the ligand, is capable of selectively recognising GD2-positive cells, and of binding them. Furthermore, thanks to the optional presence of a large number of photosensitiser molecules, the phage enables the selective elimination of the GD2-positive cells. In fact, when irradiated with light, the photosensitiser is capable of generating ROS, thus favouring the destruction of the cells to which the phage is bound.

Therefore, the phage can be used as a medicament, e.g. in photodynamic therapy (PDT) for the treatment of the herein mentioned diseases, in particular of neuroblastoma.

The present invention relates also to the phage or to the composition as described above for use as a medicament, preferably in PDT.

The present invention also refers to a vaccine comprising the bacteriophage or composition according to the invention.

The phage or the composition is preferably administered in association/in combination with light, also at a low intensity (<0.03 W/cm²) preferably with an intensity greater than 0.05 W/cm².

The present invention also refers to the phage or to the composition described above for use in the treatment or follow-up of a tumour which express GD2, preferably a malignant tumour, preferably neuroblastoma.

The present invention also refers to the phage or to the composition described above for use in the imaging and detection of a tumor which expresses GD2, preferably a malignant tumour, preferably neuroblastoma.

In one embodiment, the phage. or the composition that comprises it, is taken in association or in combination with other molecules, in particular with at least one antitumoral.

The phage or the composition that comprises it may be administered in association or in combination with a surgical treatment, preferably for the treatment of a malignant tumor, more preferably before or after an operation to remove the malignant tumour.

A further aspect of the present invention relates to a method for the treatment or follow-up of a tumor, preferably a malignant tumor. Said method comprises at least a step of administering an effective amount of a phage or of a composition that comprises it, as described above, to an individual affected by a tumor.

The phage or the composition, is preferably administered in association/in combination with light, also at a low intensity (<0.3 W/cm²), preferably with an intensity greater than 0.05 W/cm².

In one embodiment, the phage or the composition that comprises it is taken in association or in combination with other molecules, in particular antitumorals.

In one embodiment, the phage or the composition that comprises it is administered in association or in combination with a surgical treatment, preferably for the treatment of a malignant tumour, more preferably before or after an operation to remove the malignant tumour.

The anti-GD2 therapy preferably comprises the bacteriophage or composition as described above.

The present invention will now be illustrated by means of non-limiting examples, with reference to the following figures.

FIG. 1. Scheme 1. Orthogonal engineering of M13 phage pIII fused with ScFv (14G2a) retarget GD2 receptor (M13_{GD2}) followed by chemical conjugation of RB, generating M13_{GD2}-RB. Retargeting GD2 positive cell lines and GD2 negative cell lines with photodynamic therapy and fluorescence imaging.

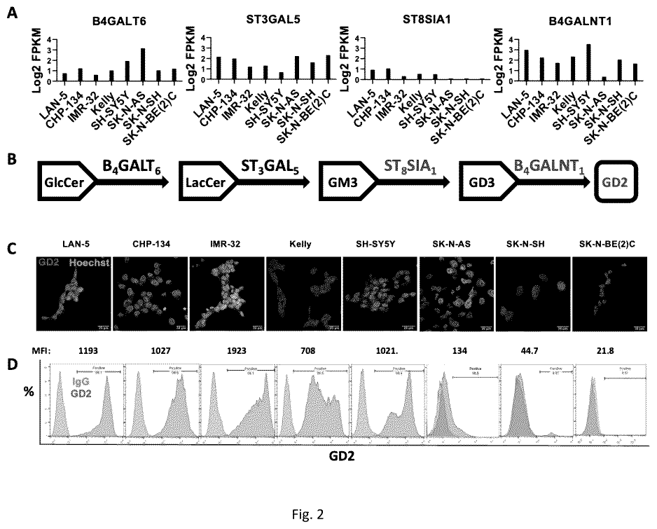

FIG. 2. Characterization of NB cell lines GD2 expression. Genes involved in biosynthesis pathway of GD2. A) B4GALT6, ST3GAL5 and B4GALNT1 are highly expressed in all panel of cell line, while ST8SIA1 that convert GM3 into GD3 is poorly expressed in SK-N-AS, SK-N-SH and SK-N-BE2C. B) Biosynthesis pathway of GD2C) Immunofluorescent Staining of GD2 in 8 NB cell lines. GD2 is expressed in LAN-5, CHP-134, IMR-32, Kelly, SH-SY5Y (green), nuclei were stained with Hoechst 33342 (blue). All scale bars are 10 μm. D) Flowcytometry analysis of GD2 by antiGD2 antibody followed by FITC conjugate mouse IgG. Blue bars show the GD2 expression while red bar shows the cell stained with isotype control IgG.

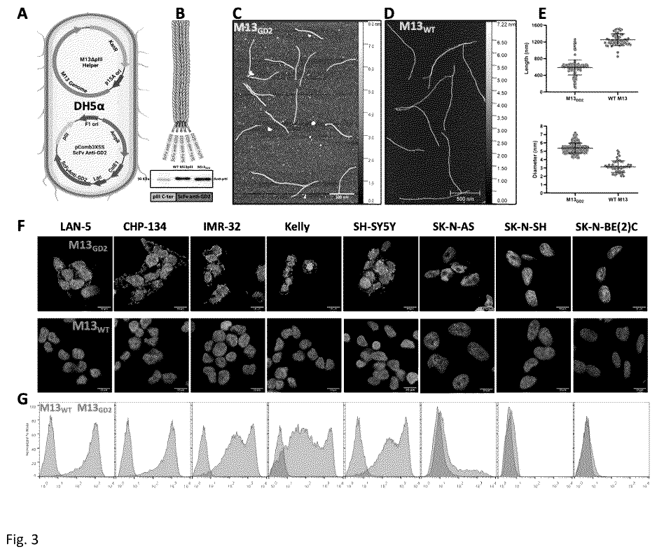

FIG. 3. M13_{GD2} refactored phage production, morphological characterization, and specificity. A) For phage production, Dh5a co-transformed with pComb3xss-ScFv anti-GD2 encoding pIII fused with ScFv against GD2 and AMP resistance (blue cassette), while Ml3pIII helper plasmid encodes for all the M13 protiens (green cassette) with Kanamycin resistance (orange cassette) except pIII, generating M13_{GD2}. B). Confirmation of pIII fused with ScFv by western blotting with anti-pIII antibody incorporated in M13_{GD2}. C) AFM micrograph of purified M13_{GD2} compared to D) AFM micrograph of purified M13 wild-type. E) Size distribution of M13_{GD2} and M13 determined by AFM, each single dot represents a single phage, mean length for M13_{GD2} is 583 nm while mean diameter is 5.37 nm. F) validation of specificity and retargeting of M13_{GD2}, as compared to wild type M13, on panel of cell line indicating strong binding of M13_{GD2} to GD2 positive cells, while no binding to GD2 negative cells, immunostaining: pVIII of M13_{GD2} and M13 (Magenta) nuclei (Hoechst, cyan; all images were acquired by 40×, Nikon Eclipse Ti2 confocal microscope. All scale bar are 10 m), G) Verification of M13_{GD2} and M13 wildtype phage retargeting to NB cell lines by flow cytometry (M13_{GD2}, Magenta; M13 wt blue).

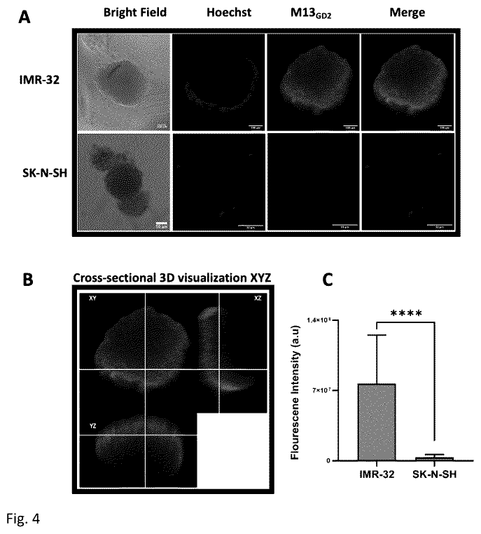

FIG. 4: M13_{GD2} mobility, penetration. A) Representation of M13_{GD2} targeting in GD2-positive IMR-32 and GD2-negative SK-N-SH generated spheroids, no internalization of phages has been seen in GD2⁻ spheroids while excellent penetration could be observed in GD2⁺ spheroids, B) 3D visualization of IMR-32 spheroid model. C) MIF of phage retargeting GD2⁺ IMR-32 and GD2⁻ SK-N-SH spheroids, all images were acquired by 40×, Nikon eclipse Ti2 confocal microscope. scale bar of IMR-32 is 10 um While For SK-N-SH is 50 um).

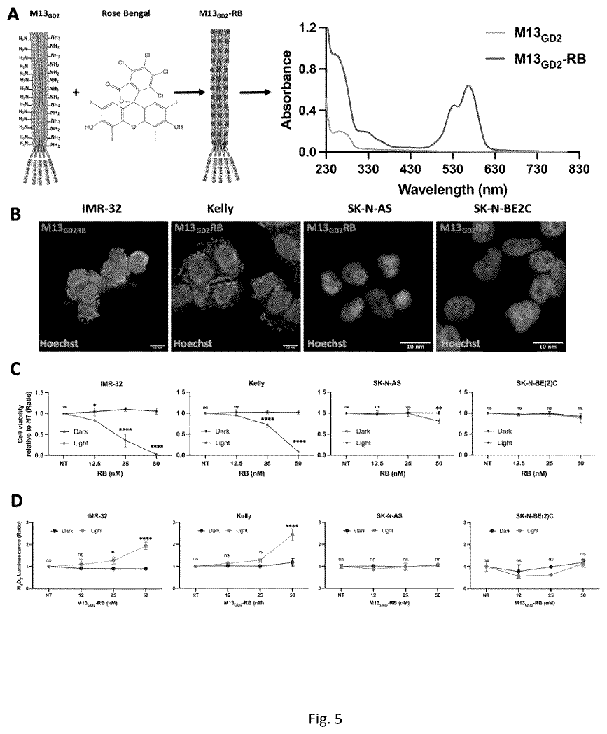

FIG. 5: Conjugation of M13_{GD2} with RB, retargeting and photodynamic killing. A) M13_{GD2} pVIII were conjugated with RB, green line represents the nude phage M13_{GD2} absorbance spectrum, while the red line represents the RB-conjugated phages (M13_{GD2}RB) absorbance spectrum B) The conjugated phages were tested on a panel of cell lines for their targeting specificity, immunostaining: pVIII of M13_{GD2}RB (Cy3: Magenta) nuclei (Hoechst, Cyan) C) Survival rates determined by MTT assay of two GD2-positive and two GD2-negative cell lines irradiated with low intensity white light 24 hours after PDT treatment with M13_{GD2}RB, controls were kept in dark. Data are presented as the mean±SD of independent quadruplicates (n=4). Significance difference of the light and dark are described as not significance=ns (p>0.05), * (P<0.05), ** (P<0.01), **** (P<0.0001). D) Extracellular Reactive oxygen produced were determined by ROS Glo assay with two GD2-positive and two-GD2-negative cell lines irradiated with low intensity white light 1 hour after PDT treatment with M13_{GD2}RB, controls were kept in dark. Data are presented as the mean±SD of four independent experiments. Significance difference of the light and dark are described as not significance=ns (p>0.05), * (P<0.05), ** (P<0.01), **** (P<0.0001).

### DETAILED DESCRIPTION OF THE INVENTION

### Materials and Methods

### Cell Culture

Human NB cell lines LAN-5, CHP-134, Kelly and SK-N-SH were cultured and maintained in RPMI 1640, with supplementation of heat-inactivated fetal bovine serum 10% (FBS) (cat.no. F9665. Sigma Aldrich), L-Glutamine 1% (200 mM) and penicillin-streptomycin 1% (100 U/ml), while IMR-32, SH-SY5Y and SK-N-BE(2)C were propagated in Dulbecco's Modified Eagle Medium (DMEM)-high Glucose (cat no. 6429, sigma Aldrich) with 10% FBS, penicillin-streptomycin 1% (100 U/ml). All cell lines were incubated in a humidified incubator containing 5% CO₂ at 37° C.

### Immunofluorescence of GD2

Initially, glass coverslips were placed in 6 wells plate and coated with collagen containing 0.02% acetic acid normal, leaving for one hour at Room Temperature (RT). Gently the coverslips were washed with Phosphate Buffer Saline (PBS) twice followed by cells (LAN-5, CHP-134, IMR-32, Kelly, SH-SY5Y, SK-N-AS, SK-N-SH, SK-N-BE(2)C) seeding and propagation overnight reaching to 80% confluency. Culture media were aspirated and washed with PBS thenceforth cells were fixed with 4% Paraformaldehyde (PFA-PBS1x) at RT for 15 minutes. After which cells were briefly washed with PBS-0.05% Tween 20 and blocked with normal donkey serum (NDS) 4% for 45 min at RT and followed by primary antibody anti-GD2 14G2a overnight incubation at 4° C and next day incubated with secondary antibody conjugated with fitch for one hour at RT, nuclear staining was carried out with Hoechst 1 ug/ml for 10 minutes and coverslips were mounted in mounting media. Images were acquired by 40×, NIKON eclipse Ti2 confocal microscope and ImageJ were used for analysis.

### Flowcytometry Analysis of GD2

Expression analysis of GD2 was carried out by flowcytometry. 1 million cells of each cell line (LAN-5, CHP-134, IMR-32, Kelly, SH-SY5Y, SK-N-AS, SK-N-SH, SK-N-Be(2)c) were harvested and resuspended in cold PBS1 supplemented with FBS 1%. All cell lines were incubated for 45 minutes at RT with anti-GD2 mAb (Santa Cruz) to labeled GD2 while the isotype PK136 mAb (mouse IgG2a) were used as a negative control for GD2 expression. Subsequently, the cells were washed three times by centrifugation with cold-PBS at 400 g at 4° C. After that, cells were incubated with FITC-labeled anti-mouse IgG for 45 minutes in dark condition and washed 3 times with PBS. All phases of the experiments were carried out at 4° C. The samples were immediately analyzed by using Bio-Rad flowcytometry. For each cell line, there were minimum of 10000 events collected and analysis was performed by flowJo software.

### Cloning of Phagemid and M13_{GD2} Preparation

The single-chain fragment variable ScFv sequence recognizing GD2 was obtained from 14G2a human-mouse chimeric antibody and the VH-VL were joined by a (Gly4Ser)₃ linker. (DNA sequence used for cloning:

(SEQ ID NO: 3)
GAAGTTCAGCTGCTGCAATCTGGTCCAGAACTGGAGAAACCGGGTGCGTC
TGTTATGATCTCTTGTAAAGCATCTGGCTCCAGCTTCACCGGTTACAACA
TGAACTGGGTTCGTCAGAACATCGGCAAAAGCCTGGAATGGATCGGCGCG
ATCGACCCATACTATGGCGGCACCTCTTACAACCAGAAATTCAAAGGTCG
CGCTACGCTGACTGTCGATAAATCTTCTTCCACCGCATATATGCACCTGA
AAAGCCTGACTTCTGAAGACTCTGCCGTATACTACTGCGTTAGCGGTATG
GAATACTGGGGTCAGGGTACTTCCGTTACCGTTTCTGGTGGTGGCGGTTC
TGGTGGTGGTGGTTCTGGTGGTGGCGGTAGCGATGTTGTGATGACCCAGA
CCCCTCTGAGCCTGCCGGTATCCCTGGGTGACCAGGCTTCTATCTCTTGC
CGTTCCTCTCAGTCTCTGGTTCATCGCAATGGTAACACCTATCTGCACTG
GTATCTGCAGAAGCCGGGTCAGTCCCCAAAACTGCTGATTCATAAAGTTT
CTAACCGTTTCTCTGGTGTTCCGGACCGTTTCTCCGGTTCTGGCAGCGGT
ACTGATTTCACCCTGAAAATTAGCCGCGTCGAAGCTGAAGACCTGGGCGT
GTACTTTTGTTCTCAGTCCACCCACGTGCCGCCACTGACCTTTGGTGCTG
GTACCAAACTGGAACTGAAA.

Protein Sequence corresponding to ScFv that is displayed in fusion with the pIII C-terminal domain in the M13_{GD2} phage EVQLLQSGPELEKPGASVMISCKASGSSFTGYNMNWVRQNIGKSLEWIGAIDPYYGG TSYNQKFKGRATLTVDKSSSTAYMHLKSLTSEDSAVYYCVSGMEYWGQGTSVTVS GGGGSGGGGSGGGGSDVVMTQTPLSLPVSLGDQASISCRSSQSLVHRNGNTYLHWY LQKPGQSPKLLIHKVSNRFSGVPDRFSGSGSGTDFTLKISRVEAEDLGVYFCSQSTHV PPLTFGAGTKLELK (SEQ ID NO:4)) The synthetic gene for this ScFv was amplified using oligonucleotides (GTTTTTGAGCTCGAAGTTCAGCTGC (SEQ ID NO:1)) and (GTTTTTACTAGTTTTCAGTTCCAGTTTAGTACC (SEQ ID NO:2)) generating overhang restriction sites for SacI and SpeI at the 5′ and 3′ end of the ScFv. Further, the segment was cloned in plasmid Pcomb3xss digested with SacI and SpeI generating 14G2a phagemid followed by co-transformation of 14G2a phagemid and helper phage (M13K07 vector) in DH5a with Amp-Km selection. Positive clones were identified by colony PCR. The selected positive colonies after PCR confirmation were then propagated in 10 ml culture containing Amp-Km antibiotics supplemented with 2% glucose over night at 37° C.

Bacteria carrying 14G2a phagemid and helper phage were grown at 32° C overnight in culture containing Amp-Km resistance and 0.4 mM IPTG, reaching an OD of 2.0. Expression of phagemid was induced under P_{lac} promotor resulting a fusion of ScFv with pIII. The culture were centrifuged 10000 g for 20 min at 4° C to pellet bacteria. Gently, the supernatant was transferred to fresh bottles and additional centrifugation was carried out for 5 min to pellet the remaining bacteria and the supernatant containing M13_{GD2} were carefully transferred to fresh bottles. Next, NaCl 4% and PEG 8000 3% were added to supernatant and carefully inverted the bottles to dissolve and incubated for 90 minutes at 4° C, supernatant containing M13_{GD2}/PEG 8000/NaCl were centrifuged at 10000 g for 20 minutes at 4° C. Pelleted phages were tenderly resuspended in PBS followed with lowering the PH to 4.2 with HCL 0.5M for phages isoelectric point (IEP) and centrifuged with 14000 g for 15 min at 4° C followed by gentle resuspension of pellet in PBS. The additional IEP were performed to eliminate the endotoxins derived from the bacterial culture for downstream applications. Phage concentrations were checked by UV-Vis Spectrophotometer at 269 nm, using an extinction coefficient of ε=3.84 μM.

### Western Blotting

ScFv fused with pIII were confirmed with western blotting by electrophoresing 2·10¹⁰ M13_{GD2} phages in 10% sodium dodecyl sulfate polyacrylamide gel electrophoresis (SDS-PAGE). Transferred to nitrocellulose membrane for 2 hours at 260 mAmp at 4° C. Afterward, the membrane was blocked for 1 hour at room temperature with blocking buffer containing 2.5 percent powder milk supplemented with 0.05 percent tween 20 to prevent non-specific antibody binding. Primary Anti-m13 pIII monoclonal antibody (new England biolab) were diluted (titer, 1:400) in blocking buffer and incubated overnight at 4° C. Next 3 washes were done with milk and secondary antibody IgG anti-mouse conjugated with horseradish peroxidase HRP were diluted (titer 1:8000) and incubated for 1 hour at RT. Membrane was developed with ECL solution ((1.25 mM luminol in Tris 100 mM pH 8.8; 6.8 mM coumaric acid; 30% H2O2). CemiDoc™ (Bio-Rad) were used to acquire images.

### Characterization of M13_{GD2} and M13 Wild Type by AFM

Atomic Force Microscopy (AFM) were done with a multimode 8 AFM (Burker, USA). The hydrated phages were loaded on the surface of Freshly Cleaved Muscovite mica (Electron Microscopy Sciences, USA) for absorbtion. A 10 ul sample of concentrated phages diluted in PBS was layered on the surface of mica and let it to be absorbed for 2 min followed by AFM fluid cell was mounted and then small volume of the ultrapure water injected in the cells. ScanAsyst Fluid+ probes were used to perform imaging in liquid using Peak Force Tapping® (Bruker, U.S.A.).

Images were processed by flattering the micrograph with Nanoscope Analysis Software (ver. 1.80). Using a custom-developed MATLAB script, the (x,y,z) coordinates of the contour of hundreds of individual phages were semi-automatically digitized from micrographs (Mathworks, U.S.A.). The measurement algorithm was implemented that had previously been devised (Zuccheri et al., 2001; Sampaolese et al., 2002).

Validation of Refactored M13_{GD2} with confocal microscopy

To test the retargeting efficacy of M13_{GD2}, 1 million cells/well (LAN-5, CHP-134, IMR-32, Kelly, SH-SY5Y, SK-N-AS, SK-N-SH, SK-N-Be(2)c) were seeded overnight reaching 60% confluence and then 1.66 nM of M13_{GD2} phages were resuspended in DMEM and incubated for 45 minutes at RT followed by twice washes with washing buffer (PBS+0.05% tween 20). subsequently cells fixation was carried out by PFA 4% for 15 min at RT and gently washed thrice with washing buffer. Next, cells were blocked by blocking solution (PBS+0.05% tween 20+4% NDS). After blocking, cells were incubated with Primary antibody anti-M13 PVIII (dilution 1:300) overnight at 4° C and then FITC-labelled anti-mouse IgG. Nuclei were stained with Hoechst 33342 (1 ug/ml) for 10 min. Images were acquired by NIKON eclipse Ti2 confocal microscope and ImageJ were used for analysis. 3D model and confocal imaging

For Spheroid experiments, IMR-32 (GD2⁺) and SKNSH (GD2⁻) cells were seeded in Ultra low attachment ULA U shaped 96 wells plate. In brief, 1000 cells of IMR-32 and SK-N-SH were seeded per well in DMEM and RPMI complete medium supplemented with 10% FBS, penicillin-streptomycin 1% (100 U/ml) respectively. The ULA plates were then centrifuged with 400 g for 5 minutes to bring cells in close contact for rapid spheroid generation.

### Bioconjugation and Purification of M13_{GD2}-RB Phages

Bioconjugation of RB. RB was dissolved in DMSO to obtain a concentration 10 mM, then 1-ethyl-3-(3-dimethylaminopropyl) carbodiimide (EDC) and N-hydroxysuccinimide (NHS) were added under stirring, to obtain a final concentration of 10 mM and 15 mM, respectively. The solution was incubated for 3 hours at 25° C under constant shaking at 700 rpm (ThermoMixer HC, S8012-0000; STARLAB, Hamburg, Germany) in dark conditions. 50 μl of the activated RB solution was then added dropwise under vigorous stirring, to 1 ml of a PBS solution containing M13_{GD2} phages at a concentration 40 nM. The solution was then incubated overnight at 25° C under mild stirring condition (700 rpm).

Validation of M13_{G}D2-RB with Confocal Microscopy

Since RB has fluorescence and bioconjugate with M13_{GD2}. The M13_{GD2}-RB localization and its interaction were determined by confocal microscopy. Round coverslips were placed in 6 wells and 50000 cells (LAN-5, IMR-32, Kelly SK-N-AS, SK-N-SH) were seeded overnight reaching to 70% confluency. Next cells were incubated with M13_{GD2}-RB resuspended in complete medium for 90 min, washed two time with PBS1x and stained with Hoechst 1 ug/ml for 10 min. the coverslips were transferred to Attofluor cell chamber and 1 ml of complete medium without red phenol added to the chamber. Images were acquired with Nikon AIR1 confocal microscope and analyzed with ImageJ for fluorescence intensity.

### Photodynamic Treatment 2D Culture

For PDT we selected four cell lines including two high expressing GD2 (IMR-32, Kelly) and one very low expressing cell line (SK-N-AS) and a GD2-negative cell line (SK-N-SH). Cells were seeded for cell viability assay at density of 50000/well in 96 well plate and incubated in humidified incubator containing 5% CO₂ at 37° C after 24 hours, the medium was aspirated and adherent cells washed with PBS. Subsequently, cells were incubated with M13_{GD2}-RB starting from a concentration of 1.66 nm with two-folds serial dilution down to 0.026 nm in DMEM supplemented with 10% FBS, penicillin-streptomycin 1% (100 U/ml) for 90 minutes. Following incubation, the cells were washed three times with DMEM without red phenol (cat no. 21063029) to eliminate unbound M13_{GD2}-RB. Further, the cells were irradiated with white lamp from 30 cm distance for 15 min and in irradiation step the cells were maintained in DMEM without red phenol to allow the light to flow through readily. Immediately, after irradiation the cells medium was changed and placed in incubator for 24 hours. Control experiments were conducted under the same conditions I), cells were exposed to various concentration of M13_{GD2}-RB with irradiation (phage toxicity), II) the cells were exposed to light (Phototoxicity) III), the cells were kept in dark with M13_{GD2}-RB without irradiation (Dark phage toxicity), IV) the cells were kept in dark (Dark toxicity).

### Cell Viability Assay MTT

Cell viability was determined using the 3-[4,5-dimethylthiazole-2-yl]-2,5-diphenyltetrazolium bromide (MTT) assays after 24 hours of post-PDT. Gently, the medium was aspirated from the 96-well plate and the freshly added medium contained a final concentration of MTT 0.5 mg/mL. Cells were placed for 90 minutes for incubation in humidified incubation containing 5% CO2 at 37° C. After incubation, the insoluble product formazan produced by the reduction of MTT by NADPH dependent oxidoreductases expressed in live cell was solubilized in dimethyl sulfoxide at RT and protected from light. The absorbance was then assessed at the wavelength of 570 and 690 nm by the EnSpire Multimode plate reader (Perkin, Inspire, USA).

### Results

### GD2 Expression Characterization in NB Cell Lines

Given the literature ambiguity for GD2 expression in NB cellular models, we analysed a panel of NB cell lines for the expression of crucial genes encoding for enzymes involved in GD2 biosynthesis. The pathway for GD2 biosynthesis requires 4 enzymes, B4GALT6, ST3GAL5, ST8SIA1 and B4GALNT1 which converts GlcCer into LacCer, LacCer into GM3, GM3 into GD3 and GD3 into GD2, respectively. Among these enzymes, ST8SIA1 and B4GALNT1 are directly involved in catalysing GD2 synthesis (FIG. 2A). According to the NB cell lines RNAseq database provided by Maris et al. (Ref.), B4GALT6, ST3GAL5 and B4GALNT1 were expressed in all the panel of cell lines. However, the gene ST8SIA1, encoding for the enzyme involved in GM3 into GD2 conversion was poorly expressed in SK-N-AS, SK-N-SH and SK-N-BE (2)C compared with the other cell lines. These findings let us hypothesize that the latter cell lines could be GD2 negative (GD2−). To test this hypothesis, we performed both GD2 immunofluorescence (IF) and flow cytometry (FC) experiments to respectively have a qualitative and quantitative evaluation of GD2 expression. As expected, GD2 IF showed that LAN-5, CHP-134, IMR32, Kelly and SH-SY5Y were GD2+, while SK-N-AS, SK-N-SH and BE(2)C were GD2− (FIG. 2B). Interestingly, FC analysis revealed that LAN-5 and CHP-134 had a high homogenous expression of GD2 (99.1% MFI:1193 and 99.6% MFI:1027 respectively), IMR-32 express high GD2 in a heterogenous pattern (99.1% MFI:1923), while Kelly and SH-SY5Y showed heterogeneous expression of GD2 (99.8%+ MFI:708 and 99.4%+ MFI:1021). Moreover, Kelly and SH-SY5Y showed at least two populations of cells, expressing low and high levels of GD2. In the context of GD2− cell lines, a significant percentage of SK-N-AS showed low levels of positivity (35.4%+ MFI: 134), while SK-N-SH (4.07%+ MFI: 44.7) and SK-N-BE (2)C were negative for the GD2 staining (FIG. 2C).

### Engineering of a GD2 Target Vector Based on the M13 Bacteriophage.

To engineer a vector able to target specifically GD2-positive NB cells, we genetically modified the M13K07 phage by fusing an anti-GD2 single chain fragment variable (ScFv) to the C-terminal domain of the pIII protein, generating M13_{GD2}. The sequence of the ScFv chosen for M13_{GD2} production corresponds with the heavy and light chain's variable region of the 14.G2a antibody. We chose 14.G2a antibody since it is an FDA-approved antibody that has been extensively used for GD2 targeting immunotherapy (Dhillon, 2015). For M13_{GD2} production we co-transformed the DH5α E. coli strain with two plasmids, I) M13ΔpIII helper: encoding, all the minor and major structural proteins necessary for M13 bacteriophage assembly except pIII protein. II) Phagemid: pIII-ScFv anti-GD2: encoding the pIII protein fused with ScFV retargeting GD2 (FIG. 3A). The correct expression of the recombinant pIII protein fused with scFv was checked by western blot, observing a band of the expected molecular weight recognized by anti-M13 pIII antibody (FIG. 3B). To evaluate and compare the morphology of M13_{GD2} and wild type M13, we performed atomic-force microscopy (AFM) experiments (FIG. 3C, D). Likewise, M13_{GD2} showed intact architecture and no substantial structural abnormalities compared to the wild-type M13, confirming that the genetic modifications had no impact on the structure and integrity of the virion. In fact, M13_{GD2} showed a mean diameter of 5.337 nm (Std. 0.610) and a mean length of 583.3 nm (Std. 169.6). To validate the propensity of refactored M13_{GD2} to target GD2-positive NB cell lines, we incubated different concentrations of M13_{GD2} with GD2-positive (GD2⁺) and GD2-negative (GD2⁻) cell lines and tracked the phage tropism with anti-pVIII antibody by immunofluorescence microscopy. Our results showed that the minimum concentration needed to detect phage binding of GD2⁺ cells was between 10⁹ and maximum 10¹¹ M13_{GD2}/mL. Strikingly, we verified that M13_{GD2} targets only the GD2⁺ cell lines, whereas it does not bind to GD2⁻ cells even at higher concentration of the phage 10¹¹ M13_{GD2}/mL (FIG. 3E). These results demonstrate that M13_{GD2} is a extremely specificity vector, potentially usable for drug delivery to NB cells.

### The M13_{GD2} Vector Penetrates NB Spheroids.

Poor tissue penetration of antibody-based drugs and insufficient tumor penetration of chemotherapeutics continue to be the key barrier for solid tumor therapies. To deepen the investigation of the M13_{GD2} vector on 3D models of NB, we tested M13_{GD2} on two different kinds of spheroids generated from IMR-32 (GD2⁺) and SK-N-SH (GD2⁻) cell lines. Using brightfield and fluorescence microscopy to detect M13_{GD2}, we made the surprising observation that the phage penetrated deep into the tumour spheroids of IMR-32 3D model, while no significant intratumoral accumulation was observed in SK-N-SH 3D model. (FIG. 4). The deep penetration of phage in multicellular spheroid model was not expected and was never reported before in literature. This feature of the phage vector can open a window to overcome the intratumoral diffusion resistance which is a persistent obstacle that greatly reduces the effectiveness of drug delivery and overall cancer treatment outcomes (Prabhakar et al., 2013).

The M13_{GD2} Vector Mediates Targeted Delivery of Rose Bengal Sensitizers for Photodynamic Killing of GD2⁺ Cells

To test M13_{GD2} for the capacity to deliver drugs, we chemically conjugated M13_{GD2} with Rose Bengal, a photoreactive molecule approved by the FDA as an ocular diagnostic stain and orphan anticancer drug (Demartis et al., 2021), generating M13_{GD2}-RB. After the chemical conjugation of M13_{GD2} with RB, we analyzed its UV-vis absorption spectrum to provide an accurate quantification for subsequent downstream experiments.

As described in FIG. 5(A), the conjugation reaction involves ε-amino functionalisation of M13_{GD2} proteins. This profound modification of M13_{GD2} molecules may lead to loss of binding capacity to GD2⁺ cell lines. To check the retargeting potential of M13_{GD2}-RB on GD²⁺ NB cells we stained the phage vector with an antibody against the major capsid protein pVIII as shown in FIG. 5(B). The staining results indicated that M13_{GD2}-RB maintained its specificity for GD2⁺ cells without binding to GD2⁻ cells. The maintenance of high selectivity, despite the high number of RB molecules conjugated to the phages, indicates that surprisingly the targeting molecules were not affected by decoration with RB. This represents an additional advantage over known technologies of the art involving direct conjugation of photosensitizers to the targeting molecules (e.g. antibodies, scFv, etc), since it is known to the person skilled in the art that these lose affinity for their targets when conjugated with high numbers of effector (sensitizer) molecules (Pelegrin et al., 1991; van Dongen et al., 2004). After validation of the M13_{GD2}-RB specificity, we investigate the photodynamic activity of M13_{GD2}-RB and its killing potential of two GD2⁺ cell lines (IMR-32 and Kelly), a very low expressing GD2 cell line (SK-N-AS) and GD2-negative cell line (SK-BE2C). We incubated the aforementioned cell lines with various concentration of M13_{GD2}-RB (RB nM: 50, 25, 12 corresponded to M13_{GD2} pM: 180, 90, 45). After washing away the free phages, the cells were irradiated with low power white light and controls were kept in dark. Survival rate was measured after 24 hours of cells recovery by the MTT assay. Significant photosensitization was observed in irradiated cells in a dose-dependent manner, even at picomolar concentration of M13_{GD2}-RB as compared to controls kept in the dark. Remarkably, no killing was observed on GD2-negative cells upon irradiation as well as in dark conditions (FIG. 5C). Consistently, the killing of GD2 positive cells was associated with the production of reactive oxygen species (ROS) production while no significant ROS were observed in GD2-negative cell lines (FIG. 5D). Altogether, these findings demonstrated that M13_{GD2}-RB is very efficient in killing specifically GD2⁺ cells.

### Discussion

Currently, NB has been treated with murine monoclonal anti-GD2 antibody alone or in combination with IL2 and GM-CSF had limited anti-tumor effects with side effects of pain and allergic reaction; however, in phase 1 trials, the majority of the adult patients evolved neurotoxicity in conjunction with hyperleukocytosis and HAMA (Murray et al., 1994). The insufficient delivery of the drug and its efficacy in the tumor site is still a significant challenge in combating Neuroblastoma.

In summary, we employed a novel approach to target GD2 with an orthogonal nanoarchitectonic approach (both genetically and chemically) using an engineered M13 filamentous phage. We chose ScFv from the anti-GD2 antibody (14G2a) as phage targeting moiety since it was approved by the FDA in March 2017 and is currently used for NB immunotherapy. A synthetic gene encoding ScFv anti-GD2 was fused to the minor coat pIII protein, with approximately 5 copies present at the phage distal end (Sidhu, 2001), and we anticipate that a single phage can display pentavalent pIII proteins fused with ScFv. Presumably, a single phage can retarget approximately five target sites, resulting in strong binding, whereas a single mAbs can only target two sites. Further, the M13 major capsid protein pVIII with 2700 copies makes it highly capable of cargo capacity to anchor (~5400 conjugating sites) PS and other imaging tags to the vector capsid. In future, this vector platform with high carrying drug capacity and selectively targeting moiety can overcome the hurdles of antibody conjugates therapies. The refactored phage was tested on different neuroblastoma cell lines expressing GD2 and non-expressing GD2 cell lines and we demonstrated that the M13_{GD2} is specifically targeting GD2 cells while no off-target was observed in GD2-negative cell lines. The National Cancer Institute pilot program demonstrated that GD2 (expressed in melanomas, Ewing sarcomas, retinoblastomas, osteosarcomas, small cell lung cancer and soft tissue sarcomas) is a potential target and identified as the 12th most significant cancer antigen according to its immunogenicity, therapeutic properties, oncogenicity, and other characteristics (Nazha et al., 2020).

### REFERENCES

Agostinis, P., Berg, K., Cengel, K. A., Foster, T. H., Girotti, A. W., Gollnick, S. O., . . . & Golab, J. (2011). Photodynamic therapy of cancer: an update. CA: a cancer journal for clinicians, 61(4), 250-281.
Alfaleh, M. A., Alsaab, H. O., Mahmoud, A. B., Alkayyal, A. A., Jones, M. L., Mahler, S. M., & Hashem, A. M. (2020). Phage display derived monoclonal antibodies: from bench to bedside. Frontiers in immunology, 11, 1986.
Allison, R. R. (2014). Photodynamic therapy: oncologic horizons. Future Oncology, 10 (1), 123-124.
Berois, N., & Osinaga, E. (2014). Glycobiology of neuroblastoma: impact on tumor behavior, prognosis, and therapeutic strategies. Frontiers in oncology, 4, 114.
Bonaventura, P., Shekarian, T., Alcazer, V., Valladeau-Guilemond, J., Valsesia-Wittmann, S., Amigorena, S., . . . & Depil, S. (2019). Cold tumors: a therapeutic challenge for immunotherapy. Frontiers in immunology, 10, 168.
Bortot, B., Apollonio, M., Baj, G., Andolfi, L., Zupin, L., Crovella, S., . . . & Biffi, S. (2022). Advanced photodynamic therapy with an engineered M13 phage targeting EGFR: Mitochondrial localization and autophagy induction in ovarian cancer cell lines. Free Radical Biology and Medicine, 179, 242-251.
Cao, B., Yang, M., & Mao, C. (2016). Phage as a genetically modifiable supramacromolecule in chemistry, materials and medicine. Accounts of chemical research, 49(6), 1111-1120.
Casas, A., Di Venosa, G., Hasan, T., & Batlle, A. (2011). Mechanisms of resistance to photodynamic therapy. Current medicinal chemistry, 18 (16), 2486-2515.
Chang, C., Guo, W., Yu, X., Guo, C., Zhou, N., Guo, X., . . . & Zhu, Y. (2023). Engineered M13 phage as a novel therapeutic bionanomaterial for clinical applications: From tissue regeneration to cancer therapy. Materials Today Bio, 100612.
Ciaccio, R., De Rosa, P., Aloisi, S., Viggiano, M., Cimadom, L., Zadran, S. K., . . . & Milazzo, G. (2021). Targeting oncogenic transcriptional networks in neuroblastoma: from N-Mye to epigenetic drugs. International Journal of Molecular Sciences, 22(23), 12883.
Cossu, I., Bottoni, G., Loi, M., Emionite, L., Bartolini, A., Di Paolo, D., . . . & Pastorino, F. (2015). Neuroblastoma-targeted nanocarriers improve drug delivery and penetration, delay tumor growth and abrogate metastatic diffusion. Biomaterials, 68, 89-99.
Coughlan, D., Gianferante, M., Lynch, C. F., Stevens, J. L., & Harlan, L. C. (2017). Treatment and survival of childhood neuroblastoma: evidence from a population-based study in the United States. Pediatric hematology and oncology, 34(5), 320-330.
Demartis, S., Obinu, A., Gavini, E., Giunchedi, P., & Rassu, G. (2021). Nanotechnology-based rose Bengal: A broad-spectrum biomedical tool. Dyes and Pigments, 188, 109236.
Dhillon, S. (2015). Dinutuximab: first global approval. Drugs, 75, 923-927.
Dolmans, D. E., Fukumura, D., & Jain, R. K. (2003). Photodynamic therapy for cancer. Nature reviews cancer, 3(5), 380-387.
Friedman, D. N., & Henderson, T. O. (2018). Late effects and survivorship issues in patients with neuroblastoma. Children, 5(8), 107.
Gao, Y., Xiong, X., Wong, S., Charles, E. J., Lim, W. A., & Qi, L. S. (2016). Complex transcriptional modulation with orthogonal and inducible dCas9 regulators. Nature methods, 13(12), 1043-1049.
Gunaydin, G., Gedik, M. E., & Ayan, S. (2021). Photodynamic therapy for the treatment and diagnosis of cancer—a review of the current clinical status. Frontiers in Chemistry, 9, 686303.
Josefsen, L. B., & Boyle, R. W. (2012). Unique diagnostic and therapeutic roles of porphyrins and phthalocyanines in photodynamic therapy, imaging and theranostics. Theranostics, 2(9), 916.
Joseph, T. M., Kar Mahapatra, D., Esmaeili, A., Piszczyk, L., Hasanin, M. S., Kattali, M., . . . & Thomas, S. (2023). Nanoparticles: Taking a unique position in medicine. Nanomaterials, 13(3), 574.
Louis, C. U., & Shohet, J. M. (2015). Neuroblastoma: molecular pathogenesis and therapy. Annual review of medicine, 66, 49-63.
Matthay, K. K., Maris, J. M., Schleiermacher, G., Nakagawara, A., Mackall, C. L., Diller, L., & Weiss, W. A. (2016). Neuroblastoma (Primer). Nature Reviews: Disease Primers, 2(1).
Melero, I., Hervas-Stubbs, S., Glennie, M., Pardoll, D. M., & Chen, L. (2007). Immunostimulatory monoclonal antibodies for cancer therapy. Nature Reviews Cancer, 7(2), 95-106.
Meng, Q. Y., Cong, H. L., Hu, H., & Xu, F. J. (2020). Rational design and latest advances of codelivery systems for cancer therapy. Materials Today Bio, 7, 100056.
Moore, C. M., Pendse, D., & Emberton, M. (2009). Photodynamic therapy for prostate cancer—a review of current status and future promise. Nature clinical practice Urology, 6(1), 18-30.
Murray, J. L., Cunningham, J. E., Brewer, H., Mujoo, K., Zukiwski, A. A., Podoloff, D. A., . . . & Benjamin, R. S. (1994). Phase I trial of murine monoclonal antibody 14G2a administered by prolonged intravenous infusion in patients with neuroectodermal tumors. Journal of clinical oncology, 12(1), 184-193.
Nazha, B., Inal, C., & Owonikoko, T. K. (2020). Disialoganglioside GD2 expression in solid tumors and role as a target for cancer therapy. Frontiers in Oncology, 10, 1000.
Paret, C., Ustjanzew, A., Ersali, S., Seidmann, L., Jennemann, R., Ziegler, N., . . . & Faber, J. (2022). GD2 Expression in Medulloblastoma and Neuroblastoma for Personalized Immunotherapy: A Matter of Subtype. Cancers, 14 (24), 6051.
Park, J. A., & Cheung, N. K. V. (2020). Targets and antibody formats for immunotherapy of neuroblastoma. Journal of Clinical Oncology, 38(16), 1836.
Pelegrin A, Folli S, Buchegger F, Mach J P, Wagnieres G, van den Bergh H. Antibody-fluorescein conjugates for photoimmunodiagnosis of human colon carcinoma in nude mice. Cancer. 1991 May 15; 67(10):2529-37. doi: 10.1002/1097-0142(19910515)67:10<2529::aid-cncr2820671024>3.0.co;2-b. PMID: 2015553.
Portwine, C., Rae, C., Davis, J., Teira, P., Schechter, T., Lewis, V., . . . & Barr, R. D. (2016). Health-related quality of life in survivors of high-risk neuroblastoma after stem cell transplant: a national population-based perspective. Pediatric blood & cancer, 63(9), 1615-1621.
Prabhakar, U., Maeda, H., Jain, R. K., Sevick-Muraca, E. M., Zamboni, W., Farokhzad, O. C., . . . & Blakey, D. C. (2013). Challenges and key considerations of the enhanced permeability and retention effect for nanomedicine drug delivery in oncologyEPR effect and nanomedicine drug delivery in oncology. Cancer research, 73(8), 2412-2417.
Prapa, M., Chiavelli, C., Golinelli, G., Grisendi, G., Bestagno, M., Di Tinco, R., . . . & Dominici, M. (2021). GD2 CAR T cells against human glioblastoma. NPJ precision oncology, 5 (1), 93.
Principi, N., Silvestri, E., & Esposito, S. (2019). Advantages and limitations of bacteriophages for the treatment of bacterial infections. Frontiers in pharmacology, 10, 513.
Riedmayr, L. M., Hinrichsmeyer, K. S., Karguth, N., Bohm, S., Splith, V., Michalakis, S., & Becirovic, E. (2022). dCas9-VPR-mediated transcriptional activation of functionally equivalent genes for gene therapy. Nature Protocols, 17(3), 781-818.
Sampaolese, B., Bergia, A., Scipioni, A., Zuccheri, G., Savino, M., Samori, B., & De Santis, P. (2002). Recognition of the DNA sequence by an inorganic crystal surface. Proceedings of the National Academy of Sciences, 99(21), 13566-13570.
Sidhu, S. S. (2001). Engineering M13 for phage display. Biomolecular engineering, 18(2), 57-63.
Ulfo, L., Cantelli, A., Petrosino, A., Costantini, P. E., Nigro, M., Starinieri, F., . . . & Calvaresi, M. (2022). Orthogonal nanoarchitectonics of M13 phage for receptor targeted anticancer photodynamic therapy. Nanoscale, 14(3), 632-641.
van Dongen, G. a. M. S., Visser, G. W. M. & Vrouenraets, M. B. Photosensitizer-antibody conjugates for detection and therapy of cancer. Adv. Drug Deliv. Rev. 56, 31-52 (2004).
Voeller, J., & Sondel, P. M. (2019). Advances in anti-GD2 immunotherapy for treatment of high-risk neuroblastoma. Journal of pediatric hematology/oncology, 41(3), 163.
Xu, S., Bulin, A. L., Hurbin, A., Elleaume, H., Coll, J. L., & Broekgaarden, M. (2020). Photodynamic Diagnosis and Therapy for Peritoneal Carcinomatosis: Emerging Perspectives. Cancers, 12(9), 2491.
Yeoh, S. G., Sum, J. S., Lai, J. Y., W Isa, W. H., & Lim, T. S. (2022). Potential of phage display antibody technology for cardiovascular disease immunotherapy. Journal of Cardiovascular Translational Research, 15(2), 360-380.
Zhang, T., de Waard, A. A., Wuhrer, M., & Spaapen, R. M. (2019). The role of glycosphingolipids in immune cell functions. Frontiers in immunology, 10, 90.
Zuccheri, G., Scipioni, A., Cavaliere, V., Gargiulo, G., De Santis, P., & Samori, B. (2001). Mapping the intrinsic curvature and flexibility along the DNA chain. Proceedings of the National Academy of Sciences, 98(6), 3074-3079.

1: A bacteriophage, comprising and/or expressing at least one ligand targeting a disialoganglioside (GD2),
wherein the bacteriophage is conjugated with at least one molecule selected from the group consisting of: a photosensitizer, a chemotherapeutic drug, a lipid, a fluorophore, a reporter, a contrast agent, a radionuclide, a boronated molecule, a catalyst, a polymer, a polysaccharide, a nucleic acid, a surfactant, a positively charged molecule, an amphiphilic molecule, an anti-microbial glycan, and a Toll-Like Receptor agonist.

2: The bacteriophage of claim 1, wherein the bacteriophage is selected from the group consisting of: M13, fd phage, Ff phage, T4, Qb and MS2 phage.

3: The bacteriophage of claim 1, wherein:
(a) the ligand is selected from the qroup consistinq of: an antibody or an antigen binding fragment thereof, a receptor, a nanobody, a protein, a peptide and any combination thereof; or
(b) the ligand is encoded by a nucleic acid fused to a nucleotide sequence encoding a coat protein of the bacteriophage.

4: The bacteriophage of claim 1, wherein the antigen-binding fragment comprises a single chain fragment variable (scFv), Fab, Fab′, or F(ab′)2.

5: The bacteriophage of claim 4, wherein the single-chain fragment variable (ScFv) or the single-chain fragment variable (ScFv) sequence:
(a) comprises an amino acid sequence having at least 80% identity to SEQ ID NO:4; or
(b) is encoded by a nucleic acid sequence having at least 80% of identity to SEQ ID NO:3.

6: The bacteriophage of claim 1, wherein the bacteriophage is genetically modified by fusing at least one of the ligands to a pIII protein of the bacteriophage.

7: The bacteriophage of claim 1, wherein, the bacteriophage comprises 1, 2, 3, 4, or 5 copies of the ligand.

8: The bacteriophage of claim 1, wherein the photosensitizer is selected from the group consisting of:
(a) Bacteriochlorin, Phthalocyanines, Pheophorbide A and B, Bacteriopheophorbide, Naphthalocyanine, BODIPY, Anthracycline, Tetracycline, Fluoroquinolones, Phenothiazine, Perylene, Perylenequinone, Curcumin, Quinones, Anthraquinones, Fullerenes, Endohedral Metallofullerenes, Acridines, Isoquinole alkaloids, Xanthenes, Porphyrins, Chlorins and Cyanine;
(b) xanthens, optionally rose bengal, eosin, erythrosine B, fluorescein and rhodamine, porphyirins, optionally protoporphyirin IX, hematoporphyirin, porfimer sodium and ATX-70; chlorins, preferably chlorin e6, temoporfin and verteporfin; cyanines, optionally cyanine IR-806, indocyanine green and cyanine IR-780, and combinations thereof, or
(c) porphyrins, phthalocyanines, chlorins, bacteriochlorins, phenothiaziniums, phenazines, acridines, texaphyrins, cyanines, anthracyclins, pheophorbides, sapphyrins, fullerene, halogenated xanthenes, perylenequinonoid pigments, gilvocarcins, terthiophenes, benzophenanthridines, psoralens and riboflavin.

9: The bacteriophage of claim 1, wherein the at least one molecule is covalently linked to the bacteriophage or is not covalently bound to the bacteriophage.

10: A pharmaceutical composition or medicament comprising the bacteriophage of claim 1, and at least one excipient, vehicle or diluent.

11. (canceled)

12: A method for preventing or treating a disease or disorder mediated by GD2, comprising administering to an individual in need thereof a bacteriophage of claim 1.

13: The method of claim 12, further comprising administering to the individual in need thereof:
(a) at least one antitumoral agent, optionally an antibody, a chemotherapeutic agent, a radionuclide, a hormone therapy agent or an immune checkpoint inhibitor
(b) a surgical treatment,
(c) a treatment of a malignant tumour, optionally before or after an operation to remove the malignant tumor; or
(d) an ultrasound treatment.

14. (canceled)

15: The bacteriophage of claim 2, wherein the bacteriophage is an M13 bacteriophage, or a M13K0 bacteriophage.

16: The bacteriophage of claim 3, wherein the coat protein of the bacteriophage comprises a protein pIII.

17: The bacteriophage of claim 4, wherein the antigen-binding fragment comprises: an anti-GD2 ScFv, and optionally said ScFv comprises the heavy chain variable region (VH) and light chain variable region (VL) of the 14.G2a antibody, and optionally the VH is located at N-terminal to the VL and linked to the VL via a linker peptide.

18: The bacteriophage of claim 6, wherein the at least one ligand is fused to the C-terminal domain of the pill protein of the bacteriophage.

19: The bacteriophage of claim 1, wherein the ligand comprises an anti-GD2 ScFv.

20: The method of claim 12, wherein the disease or disorder mediated by GD2 is a GD2 positive tumor or a tumor comprising at least one cell expressing GD2.

21: The method of claim 20, wherein the GD2 positive tumor or a tumor comprising at least one cell expressing GD2 is selected from the group consisting of: a brain tumor, a neuroblastoma, a glioblastoma, a medulloblastoma, a retinoblastoma, an astrocytoma, a melanoma, a B-cell lymphoma, small cell lung cancer, renal carcinoma, desmoplastic small round cell fibroma, osteosarcoma, Ewing's sarcoma, breast cancer, rhabdomyosarcoma, leiomyosarcoma, liposarcoma, fibrosarcoma, and a soft tissue sarcoma.
